(19) United States
(12) Patent Application Publication
(10) Pub. No.: US 2009/0137821 A1
(43) Pub. Date: May 28, 2009

(54) Method of making dorzolamide hydrochloride

(76) Inventors: Laszlo Zsolt Kovacs, Debrecen (HU); Csaba Szabo, Debrecen (HU); Erika Magyar Molnarne, Debrecen (HU); Adrienne Kovacsne-Mezei, Debrecen (HU); Claude Singer, Kfar Saba (IL); Judith Aronhime, Rehovot (IL)

Correspondence Address: KENYON & KENYON LLP, ONE BROADWAY, NEW YORK, NY, 10004, US

(21) Appl. No.: 12/317,438

(22) Filed: Dec. 22, 2008

Related U.S. Application Data
(62) Division of application No. 11/326,719, filed on Jan. 6, 2006.
(60) Provisional application No. 60/642,166, filed on Jan. 6, 2005.

(51) Int. Cl. C07D 335/04

(52) U.S. Cl. 549/23

(57) ABSTRACT

Processes for the preparation of dorzolamide hydrochloride and an intermediate of Formula IV,

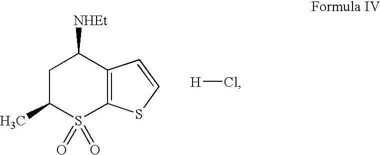

are provided.

### RELATED APPLICATIONS

The present application is a division of U.S. patent application Ser. No. 11/326,719, filed Jan. 6, 2006, which claims benefit of U.S. Provisional Patent Application No. 60/642,166, filed Jan. 6, 2005, the contents of which are incorporated herein in their entirety.

### FIELD OF THE INVENTION

The present invention is directed to methods of making dorzolamide hydrochloride.

### BACKGROUND OF THE INVENTION

Dorzolamide hydrochloride, known chemically as 5,6-dihydro-4-(S)-ethylamino-6-(S)-methyl-4H-thieno-[2,3-b]thiopyran-2-sulfonamide-7,7-dioxyde hydrochloride, is a topically effective carbonic anhydrase inhibitor useful in the treatment of ocular hypertension.

Dorzolamide hydrochloride has the structure of Formula I:

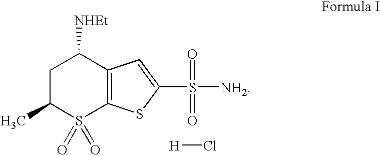

U.S. Pat. Nos. 4,677,155 and 4,797,413 disclose Dorzolamide. In the prior art synthesis of dorzolamide, a chiral hydroxysulfone is used as a starting material. The chiral hydroxysulfone starting material can be obtained using the processes disclosed in U.S. Pat. Nos. 5,157,129, 5,474,919, and 5,760,249. In the disclosed processes, the chiral hydroxysulfone is obtained by the asymmetric enzymatic reduction of the corresponding ketosulfone, or by cyclization of the chiral thienyl thiobutyric acid, obtained, in turn, from a chiral hydroxyester or lactone, and the subsequent stereospecific reduction of the resulting ketone.

Processes for the preparation of dorzolamide hydrochloride are described in U.S. Pat. Nos. 4,797,413, 5,157,129, and 5,688,968 and in U.S. Patent Application Publication No. 2003/0220509. The disclosed processes involve conversion of a hydroxysulfone to the corresponding acetamidosulfone by a Ritter reaction with retention of configuration, followed by introduction of a sulfonamido group, and the subsequent reduction of the amido group to an amine, providing the desired product.

The process disclosed in U.S. Pat. No. 4,797,413 includes activation of the 4-hydroxy group of the sulfonaminated hydroxysulfone with tosyl chloride and the introduction of the desired alkylamino group by nucleophilic substitution, resulting in all diastereomeric products, which must be separated and resolved. As a result, at least 75 percent of the product is lost when the desired product is the more active enantiomer.

There is a need in the art for a new process for the preparation of Dorzolamide and salts thereof.

### SUMMARY OF THE INVENTION

In one embodiment, the present invention is directed to a process for the preparation of a protected derivative of Formula II, comprising: protecting the hydroxy group of 5,6-dihydro-4-(R)-hydroxy-6-(S)-methyl-4H-thieno-[2,3-b]thiopyran 7,7-dioxide, having the structural Formula II

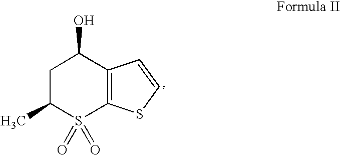

with a sulfonic acid derivative, in the presence of an organic base and a polar aprotic organic solvent, to obtain a protected derivative.

In another embodiment, the present invention is directed to a process for the preparation of a compound of Formula IV,

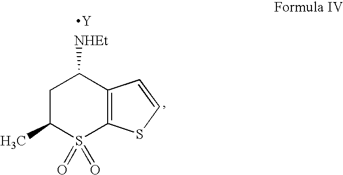

comprising: aminating the protected derivative of formula II with an alkyl amine and an acid in the presence of a solvent selected from the group consisting of a polar aprotic organic solvent, water and a mixture thereof, to give 5,6-dihydro-4-(S)-ethylamino-6-(S)-methyl-4H-thieno-[2,3-b]thiopyran 7,7-dioxide salt of Formula IV, where Y is an organic or inorganic acid moiety. Preferably Y is selected from the group consisting of acetic acid, fumaric acid, tartaric acid, sulfuric acid, hydrochloric acid, and hydrobromic acid. More preferably, the acid is HCl.

In another embodiment, the present invention is directed to a process for preparing dorzolamide salt of formula I, where Y is as defined above,

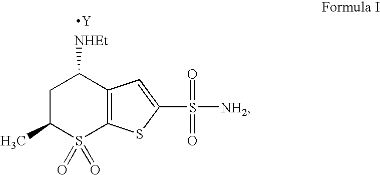

comprising: sulfonamidating of the compound of Formula IV by combining the compound of Formula IV with fuming sulfuric acid or chlorosulfonic acid, chlorinating the sulfonylated intermediate by the addition of inorganic chlorinated agent, evaporating the inorganic chlorinated agent from the reaction mixture, adding a polar aprotic organic solvent, adding a base and afterwards adding an acid until dorzolamide salt compound of Formula I is obtained.

In another embodiment, the present invention is directed to a process of purifying dorzolamide salt by dissolving the dorzolamide salt in water, adding a base until a basic slurry is obtained, extracting the basic slurry with an aprotic polar organic solvent, which is immiscible in water, until two phases are obtained, separating the organic phase, concentrating the organic phase to obtain a residue of dorzolamide base, and cooling the residue.

In another embodiment, the present invention is directed to a process of purifying dorzolamide salt by combining dorzolamide base with an acid and with a polar aprotic organic solvent to obtain an acidic slurry, cooling the slurry to obtain a precipitate of dorzolamide salt, and recovering the dorzolamide salt.

In another embodiment, the present invention is directed to a process of purifying dorzolamide salt by dissolving the dorzolamide salt in water, adding a base until a basic slurry is obtained, extracting the basic slurry with an aprotic polar organic solvent, which is immiscible in water, until two phases are obtained, separating the organic phase, concentrating the organic phase to obtain a residue of dorzolamide base, cooling the residue, combining the residue with an acid and with a polar aprotic organic solvent to obtain an acidic slurry, cooling the slurry to obtain a precipitate of dorzolamide salt, and recovering the dorzolamide salt.

In yet another embodiment, the present invention is directed to a process for the preparation of a dorzolamide salt of structural Formula I, where Y is as defined above.

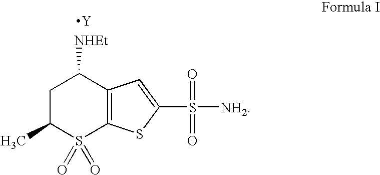

The process comprises:

(a) protecting the hydroxy group of 5,6-dihydro-4-(R)-hydroxy-6-(S)-methyl-4H-thieno-[2,3-b]thiopyran 7,7-dioxide, having the structural Formula II

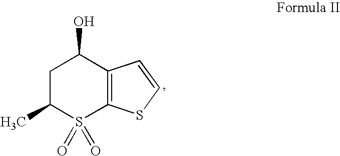

with a sulfonic acid derivative, in the presence of an organic base and a polar aprotic organic solvent, to obtain a protected derivative;

(b) aminating the protected derivative of formula II with an alkyl amine and an acid in the presence of a solvent selected from the group consisting of a polar aprotic organic solvent, water and a mixture thereof, to give 5,6-dihydro-4-(S)-ethylamino-6-(S)-methyl-4H-thieno-[2,3-b]thiopyran 7,7-dioxide salt of Formula IV, where Y is as defined above;

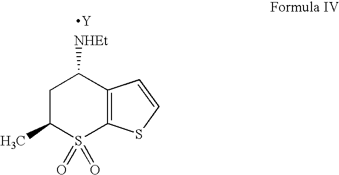

and

(c) sulfonamidating of the compound of Formula IV by combining the compound of Formula IV with fuming sulfuric acid or chlorosulfonic acid, chlorinating the sulfonylated intermediate by the addition of inorganic chlorinated agent, evaporating the inorganic chlorinated agent from the reaction mixture, adding a polar aprotic organic solvent, adding a base, and, afterwards, adding an acid until dorzolamide salt compound of Formula I is obtained;

(d) purifying the dorzolamide salt compound of Formula I; and

(e) recovering the dorzolamide salt compound of Formula I.

In another embodiment, the present invention is directed to a process for the preparation of a dorzolamide salt of structural Formula I, where Y is as defined above,

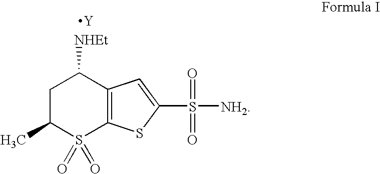

comprising obtaining a protected compound of formula II by the method described above and converting it to a compound of formula I.

In another embodiment, the present invention is directed to a process for the preparation of a dorzolamide salt of structural Formula I, where Y is as defined above,

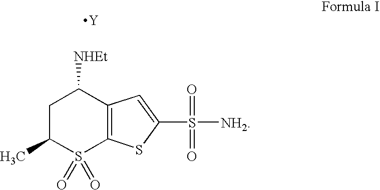

comprising obtaining a compound of formula IV by the method described above and converting it to a compound of formula I.

In yet another embodiment, the present invention is directed to a process for the preparation of a dorzolamide salt of structural Formula I, where Y is as defined above,

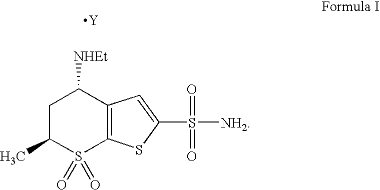

Comprising: protecting the hydroxy group of 5,6-dihydro-4-(R)-hydroxy-6-(S)-methyl-4H-thieno-[2,3-b]thiopyran 7,7-dioxide, having the structural Formula II

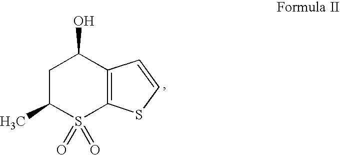

with a sulfonic acid derivative, in the presence of an organic base and a polar aprotic organic solvent, adding an alkyl amine and an acid in the presence of a solvent selected from the group consisting of a polar aprotic organic solvent, water and a mixture thereof, adding fuming sulfuric acid or chlorosulfonic acid, adding an inorganic chlorinated agent, evaporating the inorganic chlorinated agent from the reaction mixture, adding a polar aprotic organic solvent, adding a base, afterwards adding an acid until dorzolamide salt compound of Formula I is obtained, purifyng the dorzolamide salt compound of Formula I and recovering the dorzolamide salt compound of Formula I.

In another embodiment, the present invention is directed to an intermediate of the dorzolamide salt having the formula (formula III):

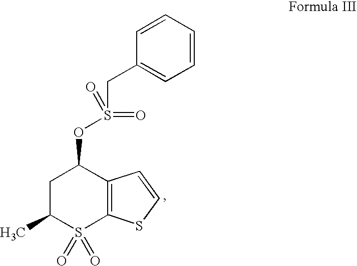

In another embodiment, the present invention is directed to an intermediate of the dorzolamide hydrochloride having the formula (formula IV):

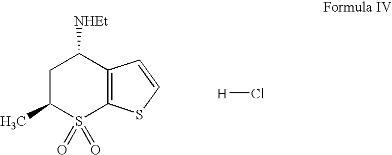

### BRIEF DESCRIPTION OF THE DRAWING

FIG. 1 illustrates the characteristic XRD pattern of the compound of Formula IV.

### DETAILED DESCRIPTION OF THE INVENTION

As used herein, the term “fuming sulfuric acid” refers to a sulfuric acid containing 10-25% free SO₃.

The present invention provides a process for the preparation of dorzolamide and salts thereof that uses a chiral starting material, thus avoiding the need for an optical resolution process to obtain the final product. The process includes transformation of one intermediate to another in a process that is almost one pot, hence isolation of only one intermediate is required. Moreover, the process can be adapted to industrial scale.

In one embodiment, the present invention is directed to a process for the preparation of a protected derivative of Formula II, comprising: protecting the hydroxy group of 5,6-dihydro-4-(R)-hydroxy-6-(S)-methyl-4H-thieno-[2,3-b]thiopyran 7,7-dioxide, having the structural Formula II

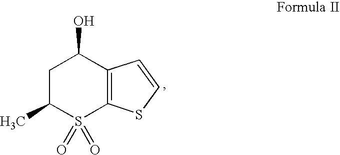

with a sulfonic acid derivative, in the presence of an organic base and a polar aprotic organic solvent, to obtain a protected derivative.

Preferably, the sulfonic acid derivative is arylsulfonyl or alkylsulfonyl chloride. More preferably, the arylsulfonyl chloride is benzylsulfonyl chloride, tosyl chloride or toluenesulfonyl chloride. Most preferably, the arylsulfonyl chloride is benzylsulfonyl chloride. When the arylsulfonyl chloride is benzylsulfonyl, the obtained protected compound is a compound of Formula III.

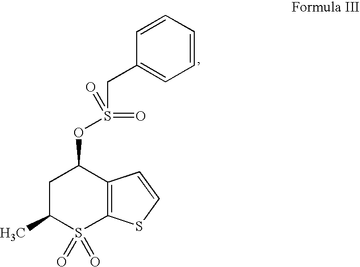

Preferably, the process is performed at a temperature of up to about 0° C. More preferably, the process is performed at a temperature of from about −30° to about 0° C. for about 2 to about 4 hours. Preferably, the organic base is selected from the group consisting of pyridine, triethylamine, and N,N-diisipropylethylamine. More preferably, the organic base is triethylamine. Preferably, the polar aprotic organic solvent is selected from the group consisting of acetone, dioxane, acetonitrile, tetrahydrofuran, ethyl acetate, 2-methyltetrahydrofuran, and pyridine. More preferably, the polar aprotic organic solvent is tetrahydrofuran or ethyl acetate. After the addition of the base, the sulfonic acid derivative, and the polar aprotic organic solvent, the reaction mixture is stirred and triethylamine HCl salt may be obtained. Preferably, the obtained triethylamine HCl salt is filtered. Following filtration, the protected compound is preferably used in the next step of the synthesis without further processing.

In another embodiment, the present invention is directed to a process for the preparation of a compound of Formula IV, where Y is as defined above,

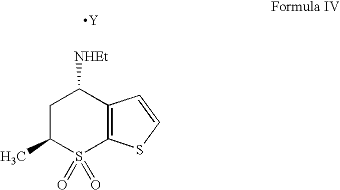

comprising: aminating the protected derivative of formula II with an alkyl amine and an acid in the presence of a solvent selected from the group consisting of a polar aprotic organic solvent, water and a mixture thereof, to give 5,6-dihydro-4-(S)-ethylamino-6-(S)-methyl-4H-thieno-[2,3-b]thiopyran 7,7-dioxide salt of Formula IV.

Preferably, the amination is carried out at a temperature of about 20° C. to about 30° C. for about 16 hours to about 20 hours. Preferably, the alkyl amine is ethyl amine. Preferably, the acid is an organic acid or an inorganic acid. Preferably, the organic acid is selected from the group consisting of acetic acid, fumaric acid, and tartaric acid. Preferably, the inorganic acid is selected from the group consisting of sulfuric acid, hydrochloric acid, and hydrobromic acid. More preferably, the inorganic acid is HCl. Preferably, the polar aprotic organic solvent is selected from the group consisting of acetone, dioxane, acetonitrile, tetrahydrofuran, ethyl acetate, 2-methyltetrahydrofuran, and pyridine. More preferably, the polar aprotic organic solvent is tetrahydrofuran or ethyl acetate. Preferably, the acid is added until a pH of about 2 to about 2.5 is obtained. Preferably, the acid is added to the solution formed with alkyl amine and the solvent. Preferably, the solution is heated to a temperature of about 40° C. prior to the addition of HCl. Preferably, the solvent is removed from the reaction mixture. Preferably, the reaction mixture, containing alkyl amine, an acid and a solvent, is cooled to a temperature of about −15° C. to about 10° C., more preferably, to a temperature of about −8° C.

In another embodiment, the present invention is directed to a process for preparing dorzolamide salt of Formula I, where Y is as defined above

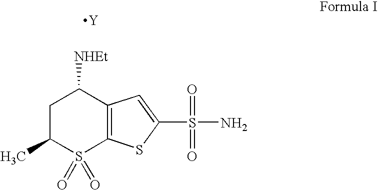

comprising: sulfonamidating of the compound of Formula IV by combining the compound of Formula IV with fuming sulfuric acid or chlorosulfonic acid, chlorinating the sulfonylated intermediate by the addition of inorganic chlorinated agent, evaporating the inorganic chlorinated agent from the reaction mixture, adding a polar aprotic organic solvent, adding a base and afterwards adding an acid until the dorzolamide salt compound of Formula I is obtained.

Preferably, the compound of Formula IV undergoes sulfonylation at a temperature of about −10° C. to about 25° C. for about 2 to about 24 hours. Preferably, the resulting sulfonylated intermediate is not isolated, but is used directly in the next stage of step, which comprises chlorination of the sulfonylated intermediate by the addition of inorganic chlorinated agent at a temperature of from about −10° to about 25° C. Preferably, the inorganic chlorinated agent is selected from the group consisting of thionyl chloride, SO₂Cl₂, PCl₃, and POCl₃. The sulfonylated intermediate can be isolated by addition of an alcohol, such as n-butanol, and then reacted with an inorganic chlorinated agent. Preferably, after the addition of the inorganic chlorinated agent, the reaction mixture is heated to a temperature of about 60° C. to about 65° C. Preferably, after the chlorination is completed, the excess of inorganic chlorinated agent is removed from the reaction mixture, preferably, by evaporation. Preferably, a residue is obtained after the evaporation. Preferably, after the evaporation of the inorganic chlorinated agent, a residue or diluted residue is obtained. Preferably, the polar aprotic organic solvent is selected from the group consisting of acetone, dioxane, acetonitrile, tetrahydrofuran, ethyl acetate, 2-methyltetrahydrofuran, and pyridine. More preferably, the polar aprotic organic solvent is tetrahydrofuran or ethyl acetate. Preferably, the base is added at a temperature of about −15° C. to about 30° C. Preferably, the residue or diluted residue is added to the base. Preferably, the base is an organic base or an inorganic base. Preferably, the organic base is ammonia. Preferably, the ammonia is an aqueous ammonia. Preferably, the inorganic base is selected from the group consisting of NaOH, KOH, K₂CO₃, and Na₂CO₃. Preferably, the organic solvent is removed from the reaction mixture. Optionally, the acid is added in an alcohol such as ethanol. Preferably, the dorzolamide salt is dorzolamide HCl.

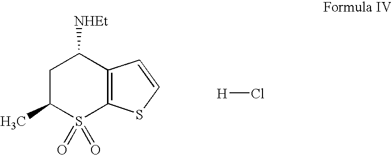

In another embodiment, the present invention is directed to a process of purifying dorzolamide salt by dissolving the dorzolamide salt in water, adding a base until a basic slurry is obtained, extracting the basic slurry with an aprotic polar organic solvent, which is immiscible in water, until two phases are obtained, separating the organic phase, concentrating the organic phase to obtain a residue of dorzolamide base, and cooling the residue.

Preferably, the dorzolamide hydrochloride is dissolved in water at a temperature of about 20° C. to about 25° C. Preferably, the base is an organic base or an inorganic base. Preferably, the organic base is ammonia. Preferably, the ammonia is an aqueous ammonia. Preferably, the inorganic base is selected from the group consisting of NaOH, KOH, K₂CO₃, and Na₂CO₃. Preferably, the base is added until a pH of about 8.0 to about 8.5 is obtained. Preferably, the aprotic polar organic solvent, which is immiscible in water, is selected from the group consisting of isobutyl acetate, ethyl acetate, and dichloromethane. More preferably, the aprotic polar organic solvent, which is immiscible in water, is ethyl acetate. Optionally, the concentration continuous until a dry dorzolamide base is obtained. Optionally, the concentration continuous until a diluted dorzolamide base is obtained. Preferably, the residue is cooled to a temperature of about 10° C. to about 30° C., more preferably, to a temperature of about 20° C. to about 25° C.

In another embodiment, the present invention is directed to a process of purifying dorzolamide salt by combining dorzolamide base with an acid and with a polar aprotic organic solvent to obtain an acidic slurry, cooling the slurry to obtain a precipitate of dorzolamide salt, and recovering the dorzolamide salt.

Preferably, the acid is an organic acid or an inorganic acid. Preferably, the organic acid is selected from the group consisting of acetic acid, fumaric acid, and tartaric acid. Preferably, the inorganic acid is selected from the group consisting of sulfuric acid, hydrochloric acid, and hydrobromic acid. More preferably, the inorganic acid is HCl. Preferably, the slurry is cooled to a temperature of about 0° C. to about 4° C. Preferably, the cooled slurry is stirred for about at least 4 hours, more preferably, for about 5 hours. Preferably, the acid is added in C₁ to C₄ alcohol. Preferably, the C₁ to C₄ alcohol is ethanol. Preferably, after cooling the slurry, the slurry is filtered until obtaining a precipitate. Preferably, the precipitate is recovered by washing it with a polar aprotic organic solvent, and drying it at a temperature of about 55° C. to about 60° C.

In another embodiment, the present invention is directed to a process of purifying dorzolamide salt by dissolving the dorzolamide salt in water, adding a base until a basic slurry is obtained, extracting the basic slurry with an aprotic polar organic solvent, which is immiscible in water, until two phases are obtained, separating the organic phase, concentrating the organic phase to obtain a residue of dorzolamide base, cooling the residue, combining the residue with an acid and with a polar aprotic organic solvent to obtain an acidic slurry, cooling the slurry to obtain a precipitate of dorzolamide salt, and recovering the dorzolamide salt.

In yet another embodiment, the present invention is directed to a process for the preparation of a dorzolamide salt of structural Formula I, where Y is as defined above,

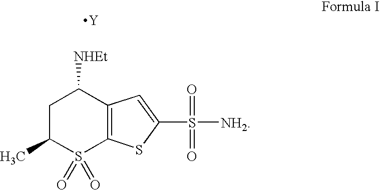

The process comprises:

(a) protecting the hydroxy group of 5,6-dihydro-4-(R)-hydroxy-6-(S)-methyl-4H-thieno-[2,3-b]thiopyran 7,7-dioxide, having the structural Formula II

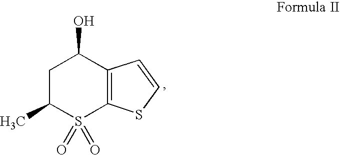

with a sulfonic acid derivative, in the presence of an organic base and a polar aprotic organic solvent, to obtain a protected derivative;

(b) aminating the protected derivative of formula II with an alkyl amine and an organic salt in the presence of a solvent selected from the group consisting of a polar aprotic organic solvent, water and a mixture thereof, to give 5,6-dihydro-4-(S)-ethylamino-6-(S)-methyl-4H-thieno-[2,3-b]thiopyran 7,7-dioxide salt of Formula IV, where Y is as defined above;

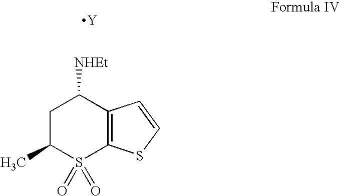

and

(c) sulfonamidating of the compound of Formula IV by combining the compound of Formula IV with fuming sulfuric acid or chlorosulfonic acid, chlorinating the sulfonylated intermediate by the addition of inorganic chlorinated agent, evaporating the inorganic chlorinated agent from the reaction mixture, adding a polar aprotic organic solvent, adding a base and afterwards adding an acid until dorzolamide salt compound of Formula I is obtained;

(d) purifying the dorzolamide salt compound of Formula I; and

(e) recovering the dorzolamide salt compound of Formula I.

The preferred reaction of the compound of Formula II with the sulfonic acid derivative in step (a) provides a sulfonic acid protecting group. Preferably, sulfonic acid derivative is arylsulfonyl or alkylsulfonyl chloride. More preferably, the arylsulfonyl chloride is benzylsulfonyl chloride, tosyl chloride or toluenesulfonyl chloride. Most preferably, the arylsulfonyl chloride is benzylsulfonyl chloride. When the arylsulfonyl chloride is benzylsulfonyl, the obtained protected compound is a compound of Formula III.

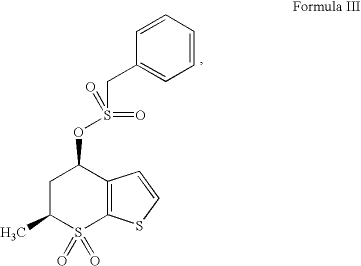

Step (a) is preferably performed at a temperature of up to about 0° C. More preferably, step (a) is performed at a temperature of from about −30° to about 0° C. for about 2 to about 4 hours. Preferably, the organic base is selected from the group consisting of pyridine, triethylamine, and N,N-diisipropylethylamine. More preferably, the organic base is triethylamine. Preferably, the polar aprotic organic solvent is selected from the group consisting of acetone, dioxane, acetonitrile, tetrahydrofuran, ethyl acetate, 2-methyltetrahydrofuran, and pyridine. More preferably, the polar aprotic organic solvent is tetrahydrofuran or ethyl acetate. After the addition of the base, the sulfonic acid derivative, and the polar aprotic organic solvent, the reaction mixture is stirred, and the triethylamine HCl salt may be obtained. Preferably, the obtained triethylamine HCl salt is filtered. Following filtration, the protected compound is preferably used in the next step of the synthesis without further processing.

Preferably, the amination in step b) is carried out at a temperature of about 20° C. to about 30° C. for about 16 hours to about 20 hours. Preferably, the alkyl amine is ethyl amine. Preferably, the acid is an organic acid or an inorganic acid. Preferably, the organic acid is selected from the group consisting of acetic acid, fumaric acid, and tartaric acid. Preferably, the inorganic acid is selected from the group consisting of sulfuric acid, hydrochloric acid, and hydrobromic acid. More preferably, the inorganic acid is HCl. Preferably, the polar aprotic organic solvent is selected from the group consisting of acetone, dioxane, acetonitrile, tetrahydrofuran, ethyl acetate, 2-methyltetrahydrofuran, and pyridine. More preferably, the polar aprotic organic solvent is tetrahydrofuran or ethyl acetate. Preferably, the acid is added until a pH of about 2 to about 2.5 is obtained. Preferably, the acid is added to the solution formed with alkyl amine and the solvent. Preferably, the solution is heated to a temperature of about 40° C. prior to the addition of HCl. Preferably, the solvent is removed from the reaction mixture. Preferably, the reaction mixture, containing alkyl amine, an acid, and a solvent, is cooled to a temperature of about −15° C. to about 10° C., more preferably, to a temperature of about −8° C.

Preferably, in step (c), the compound of Formula IV undergoes sulfonylation at a temperature of about −10° C. to about 25° C. for about 2 to about 24 hours. Preferably, the resulting sulfonylated intermediate is not isolated, but is used directly in the next stage of step, which comprises chlorination of the sulfonylated intermediate by the addition of inorganic chlorinated agent at a temperature of from about −10° to about 25° C. Preferably, the inorganic chlorinated agent is selected from the group consisting of thionyl chloride, SO₂Cl₂, PCl₃, and POCl₃. The sulfonylated intermediate can be isolated by addition of an alcohol, such as n-butanol, and then reacted with inorganic chlorinated agent. Preferably, after the addition of the inorganic chlorinated agent, the reaction mixture is heated to a temperature of about 60° C. to about 65° C. Preferably, after the chlorination is completed, the excess of inorganic chlorinated agent is removed from the reaction mixture, preferably, by evaporation. Preferably, a residue is obtained after the evaporation. Preferably, after the evaporation of the inorganic chlorinated agent, a residue or diluted residue is obtained. Preferably, the polar aprotic organic solvent is selected from the group consisting of acetone, dioxane, acetonitrile, tetrahydrofuran, ethyl acetate, 2-methyltetrahydrofuran, and pyridine. More preferably, the polar aprotic organic solvent is tetrahydrofuran or ethyl acetate. Preferably, the base is added at a temperature of about −15° C. to about 30° C. Preferably, the residue or diluted residue is added to the base. Preferably, the base is an organic base or an inorganic base. Preferably, the organic base is ammonia. Preferably, the ammonia is an aqueous ammonia. Preferably, the inorganic base is selected from the group consisting of NaOH, KOH, K₂CO₃, and Na₂CO₃. Preferably, the organic solvent is removed from the reaction mixture. Optionally, the acid is added in an alcohol such as ethanol. Preferably, the dorzolamide salt is dorzolamide HCl.

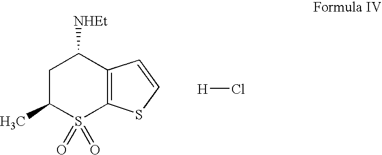

The purification in step d) of the dorzolamide salt comprises: dissolving the dorzolamide salt in water, adding a base until a basic slurry is obtained, extracting the basic slurry with an aprotic polar organic solvent, which is immiscible in water, until two phases are obtained, separating the organic phase, concentrating the organic phase to obtain a residue of dorzolamide base, cooling the residue, combining the residue with an acid and with a polar aprotic organic solvent to obtain an acidic slurry, cooling the slurry to obtain a precipitate of dorzolamide salt, and recovering the dorzolamide salt.

Optionally, the obtained dorzolamide salt may be purified by crystallizing it from water or a mixture of isopropyl alcohol-methanol (as described in example 1).

In yet another embodiment, the present invention is directed to a process for the preparation of a dorzolamide salt of structural Formula I, where Y is as defined above,

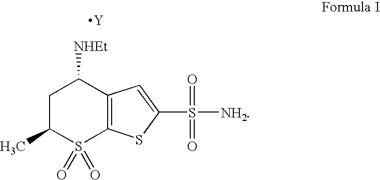

Comprising: protecting the hydroxy group of 5,6-dihydro-4-(R)-hydroxy-6-(S)-methyl-4H-thieno-[2,3-b]thiopyran 7,7-dioxide, having the structural Formula II

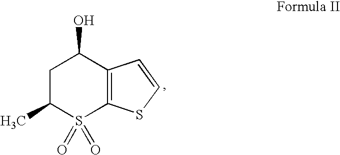

with a sulfonic acid derivative, in the presence of an organic base and a polar aprotic organic solvent, adding an alkyl amine and an acid in the presence of a solvent selected from the group consisting of a polar aprotic organic solvent, water and a mixture thereof, adding fuming sulfuric acid or chlorosulfonic acid, adding an inorganic chlorinated agent, evaporating the inorganic chlorinated agent from the reaction mixture, adding a polar aprotic organic solvent, adding a base, afterwards adding an acid until dorzolamide salt compound of Formula I is obtained, purifying the dorzolamide salt compound of Formula I and recovering the dorzolamide salt compound of Formula I.

The final purified dorzolamide hydrochloride prepared by this method does not contain more than 0.1% by weight of the corresponding 4R,6S dorzolamide.

In another embodiment, the present invention is directed to a process for the preparation of a dorzolamide salt of structural Formula I, where Y is as defined above,

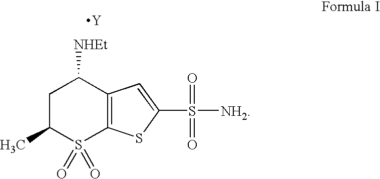

comprising obtaining a protected compound of formula II by the method described above and converting it to a compound of formula I.

In another embodiment, the present invention is directed to a process for the preparation of a dorzolamide salt of structural Formula I, where Y is as defined above,

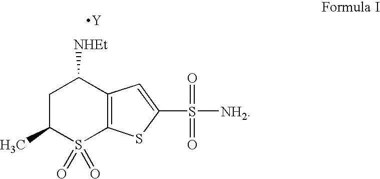

comprising obtaining a compound of formula IV by the method described above and converting it to a compound of formula I.

In another embodiment, the present invention is directed to an intermediate of the dorzolamide salt having the formula (formula III):

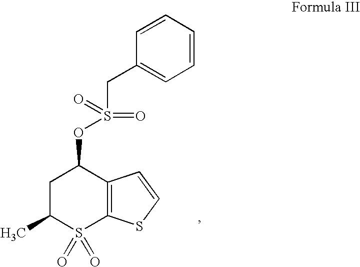

In another embodiment, the present invention is directed to an intermediate of the dorzolamide hydrochloride having the formula (formula IV):

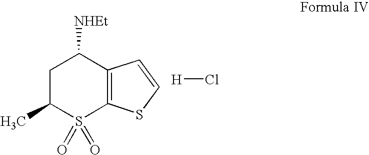

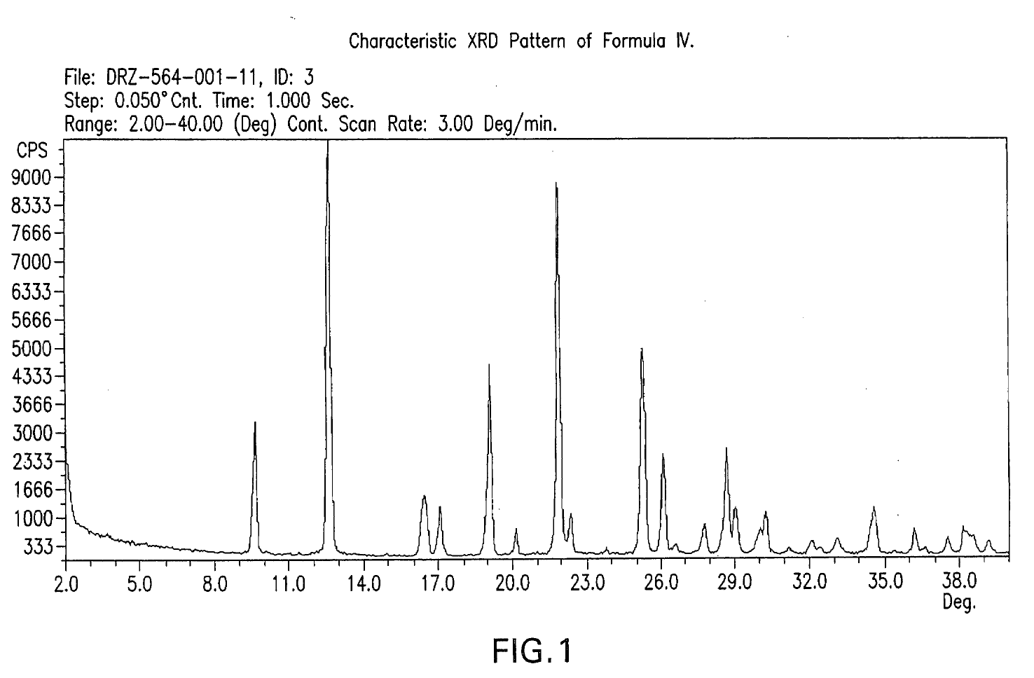

The intermediate of formula IV may be further characterized by a powder XRD pattern with peaks at 9.6, 12.6, 16.4, 17.1, 19.1, 21.9, 25.3, 26.1, 27.7 and 30.2±0.1 degrees 2θ, substantially as depicted in FIG. 1. The intermediate of formula IV may be further characterized by a ¹H NMR (DMSO-d₆) with peaks at: δ 9.74 (m, 2H), 8.12 (d, 1H), 7.65 (d, 1H), 4.68 (m, 1H), 4.20 (m, 1H), 3.16 (m, 1H), 3.05 (m, 1H), 2.78 (d, 1H), 2.53 (m, 1H), 1.37 (t, 3H), and 1.30 (t, 3H). The intermediate of formula IV may be further characterized by a ¹³C NMR (DMSO-d₆) with peaks at: δ 139.4, 138.5, 132.7, 129.7, 52.2, 50.0, 41.4, 31.8, 11.9, and 10.9. The intermediate of formula IV may be further characterized by MS: [M+H] of 246.06. The intermediate of formula IV may be further characterized by an IR (KBr): μ (cm⁻¹) 3120, 3000-2850, 2800-2500, 1560, 1540, 1522, 1300, 1264, 1140, 750, and 710.

The following non-limiting examples are merely illustrative of the preferred embodiments of the present invention, and are not to be construed as limiting the invention, the scope of, which is defined by the appended claims.

### EXAMPLE 1

A compound of Formula IV, 6-dihydro-4-(S)-ethylamino-6-(S)-methyl-4H-thieno-[2,3-b]thiopyran 7,7-dioxide hydrochloride can be synthesized according to the following scheme.

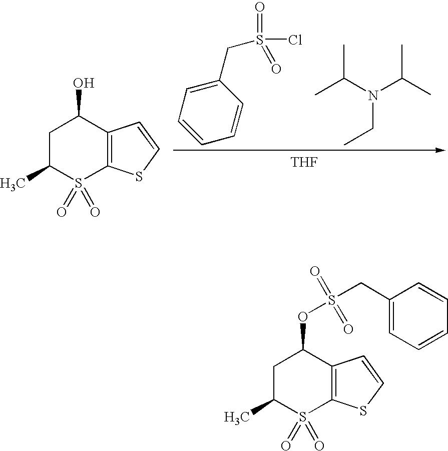

In the process of the invention, a solution of 4-(R)-hydroxy-5,6-dihydro-6-(S)-methyl-4H-thieno[2,3-b]thiopyran-7,7-dioxide, i.e., a compound of Formula II, in THF is prepared by mixing 20 g, 91.6 mmol, of the compound with 200 ml of anhydrous THF. The solution is cooled to about −20°±5° C. Triethylamine in an amount of 19.4 ml is then added gradually, while maintaining the temperature at −20°±3° C. A solution of α-toluenesulfonylchloride in an amount of 21.62 g, 109.9 mmol, in 66 ml of anhydrous THF is added in portions under an inert atmosphere, while maintaining the temperature at −18°±3° C. The reaction mixture is stirred for 2 to 2.5 hours, and the resulting triethylamine hydrochloride salt is filtered under an inert atmosphere. The cake is washed with 40 ml of cooled THF, and the combined filtrate is used immediately without further processing in the next step of the synthesis, according to the following scheme.

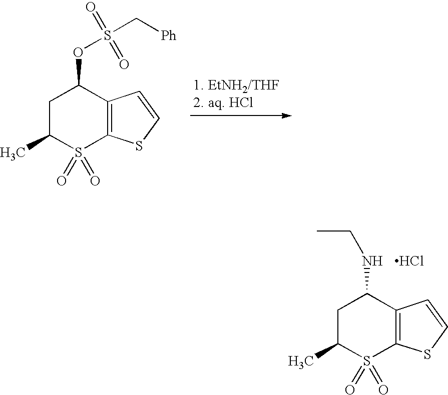

A 2 molar solution of ethylamine in THF in an amount of 674 ml is added to a cold solution of the compound of Formula III prepared according to the method discussed above in one portion at 0°±5° C. The resulting mixture is warmed to 25°±5° C., and aged for 16 to 20 hours. After aging, the mixture is cooled to −5°±5° C., and 300 ml of 4 molar aqueous hydrochloric acid is added to reduce the pH to about 2.5, while maintaining the temperature at −5°±5° C. The acidified reaction mixture is then concentrated to remove THF, and 800 ml of ethyl acetate is added. The resulting slurry is cooled to −7°±˜5° C. After stirring the suspension at −7°±5° C. for 8 to 18 hours, the resulting solid is filtered and dried at 50° C. under vacuum. The process has been shown to provide a yield of a crude aminated intermediate of Formula IV of 70 to 80 percent with a chromatographic purity of 98.5 to 99.5 percent.

The dorzolamide hydrochloride product is prepared from the aminated intermediate of Formula IV by the following scheme.

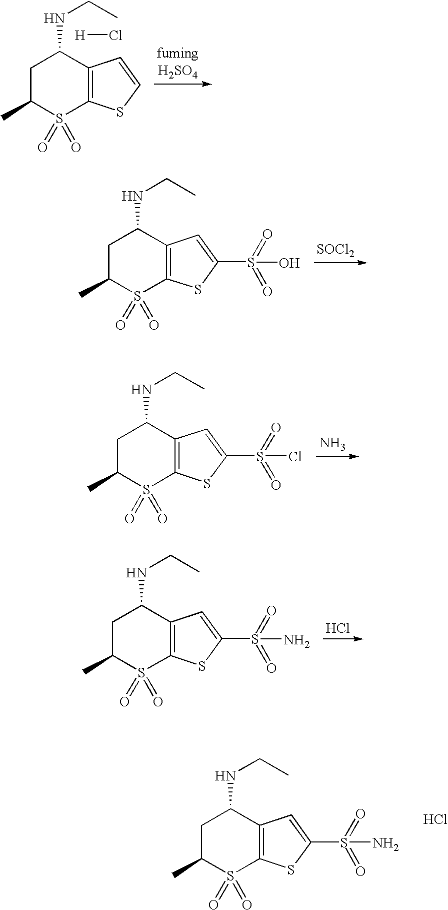

The aminated intermediate of Formula IV, in an amount of 20 g, is added in portions under an inert atmosphere to 40 ml of fuming sulfuric acid at room temperature. The reaction mixture is stirred for 2 to 3 hours at 20°±5° C. Thionyl chloride in an amount of 160 ml is then added in one portion, and the resulting mixture is refluxed for 3 to 6 hours. Excess thionyl chloride is evaporated, and the residue is stirred into a mixture of aqueous ammonia and THF (300-300 ml) in portions, under an inert atmosphere, while maintaining the temperature at −5°±5° C.

After stirring the reaction mixture for 75±15 minutes, the resulting ammonium sulfate is filtered. The cake is then washed with two 80 ml volumes of THF, the filtrate is concentrated to remove THF, and extracted with four 300 ml volumes of ethyl acetate. The organic layers are combined, concentrated to 300 ml, and stirred well, as 16 ml of concentrated hydrochloric acid is added slowly. The slurry is stirred for 40±15 minutes, filtered, and washed with two 40 ml volumes of ethyl acetate. The resulting white solid is dried under vacuum at 50° C. The process has been shown to provide a yield of crude dorzolamide HCl of 70 to 75 percent. The crude salt is recrystallized from water or a mixture of isopropyl alcohol-methanol. The amount of the 4R,6S dorzolamide (at RRt 1.09) is lower than 0.1 percent.

The sulfonylated intermediate is isolated as follows: The aminated intermediate of Formula IV, in an amount of 2 g, is added in portions under an inert atmosphere to 4 ml of fuming sulfuric acid at room temperature. The reaction mixture is stirred for 2 to 3 hours at 20°±5° C. then stirred into 240 ml of n-butanol. After stirring the suspension at −7°±5° C. for 8-18 hours, the resulting solid is filtered, washed with n-butanol, and dried at 50° C. under vacuum to give 1.80 g of the desired product in 80 percent yield. This material can be transformed to the final dorzolamide hydrochloride.

### EXAMPLE 2

### Preparation of 5,6-dihydro-4-(S)-ethylamino-6-(S)-methyl-4H-thieno-[2,3-b]thiopyran 7,7-dioxide Hydrochloride Salt (Formula IV)

Tetrahydrofuran (50 l) and triethyl amine (4.8 l) are added to 4-(R)-hydroxy-5,6-dihydro-6-(S)-methyl-4H-thieno[2,3 b]thiopyran-7,7-dioxide (5 Kg) and stirred under a nitrogen atmosphere at room temperature. The solution is cooled to −10° C. Benzylsulfonyl chloride (5.4 Kg) solved in THF (15 l) is added to the DRZ-19 THF solution in portions while maintaining the temperature below 0° C. The feeding funnel is washed with THF (2 l). The reaction mixture is stirred at 0° C. for 2-4 hours. The formed TEA HCl is filtered and the cake is washed with THF (2×10 l) Ethylamine in THF (30%, 63.7 l) is added to the filtrate and the reaction mixture is stirred at 20°-25° C. for 16 hours. Ethylamine gas prepared by heating of 70% EtNH₂ water solution (50 l) is absorbed in cooled THF (30 l). Water (20 l) is added to the reaction mixture and THF is evaporated from the filtrate at 40°±5° C. under vacuum. The residue is cooled to 20°-25° C., ethyl acetate (60 l) is added to it and stirred vigorously. After phase separation, the organic phase is washed with water (20 l). The ethyl acetate phase is heated to 40°±2° C. and hydrochloric acid (4M, ˜8-10 l) is added to it during stirring to set pH 2.0-2.5. The formed slurry is cooled to −8°±2° C. and stirred for 3 hours at this temperature. The slurry is filtered, the precipitated HCl salt is washed with ethyl acetate (30 l) and dried at 55°-60° C. under vacuum for 4-8 hours to give the desired salt (˜5 Kg).

Preparation of Dorzolamide Hydrochloride Product from the Aminated Intermediate of Formula IV

Fuming sulfuric acid (20%, 5 l) is cooled to −7°±2° C. and the aminated intermediate of Formula IV (2.5 Kg) is added to it in portions during stirring. The temperature of the reaction mixture is increased to 20°±5° C. during addition of the aminated intermediate of Formula IV. The reaction mixture is stirred for 22 hours at 20°±5° C. Thionyl chloride (20 l) is added to the stirred reaction mixture at 20±5° C. The reaction mixture is heated to 60°-65° C. and stirred for 24 hours at this temperature. The mixture is cooled back to 40±2° C. and the excess amount of thionyl chloride is evaporated at this temperature under vacuum. (The volume of the residue: ˜9 l.) The residue is cooled to −5°±2° C.

Ethyl acetate (75 l) is cooled to −10°±5° C. and the residue is added to it at this temperature. The temperature of the diluted solution: 10°-25° C. Aqueous ammonia (25%, 75 l) is cooled to −10°±5° C. and the residue is added to it at this temperature during effective stirring, while maintaining the temperature below 30° C.

The final pH: ˜11. The slurry is cooled to 0°±2° C. and stirred for 14 hours at this temperature. The formed ammonium sulfate is filtered and the cake is washed with ethyl acetate (2×20 l and 10 l). Ethyl acetate is evaporated from the filtrate at 38°±2° C. under vacuum. The residue is heated to 38°±2° C., washed with toluene (3×37.5 l) at this temperature. Water (25 l) is added to the aqueous phase, cooled to 20°-25° C., and extracted with ethyl acetate (3×75 l, 37.5 l, and 37.5 l). The collected ethyl acetate phase is concentrated to ˜100 l at 38°±2° C. under vacuum. The residue is cooled to 20°-25° C. and hydrogen chloride in ethanol (5%, 10.8 l) is added to it during stirring. The formed slurry is stirred for 1 hour at 20°-25° C. then cooled to 0°-4° C. and stirred for 5 hours at this temperature. The slurry is filtered, the precipitated HCl salt is washed with ethyl acetate (2×20 l) and dried at 55°-60° C. under vacuum for 4-8 hours to give Dorzolamide hydrochloride salt (˜2 Kg).

Crude Dorzolamide hydrochloride salt (9 Kg) is solved in water (225 l) at 20°-25° C. and the pH is set to 8.0-8.5 by addition of 25% of aqueous ammonia (2 l). The formed slurry is extracted with ethyl acetate (5×72 l). The collected ethyl acetate phase is concentrated to 180 l by vacuum distillation. The residue is cooled to 20°-25° C., ethyl acetate (45 l) and hydrogen chloride in ethanol (5%, 22.5 l) are added to it during stirring (pH: ˜1.0). The formed slurry is stirred for 1 hour at 20°-25° C. then cooled to 0°-4° C. and stirred for 5 hours at this temperature. The slurry is filtered, the precipitated HCl salt is washed with ethyl acetate (2×30 l), and dried at 55°-60° C. under vacuum for 4-8 hours to give purified Dorzolamide hydrochloride salt (˜8.2 Kg).

Purified Dorzolamide hydrochloride salt (8 Kg) dissolved in water (24 l) at 95°-105° C. and treated with active carbon (80 g). After filtration, the water solution is cooled gradually to 0°-4° C. and stirred for 3-5 hours at this temperature. The slurry is filtered, the precipitated HCl salt is washed with cooled water (2×5 l) and dried at 55°-60° C. under vacuum for 4-8 hours to give crystallized DRZ HCl salt (˜6.6 Kg).

While it is apparent that the invention disclosed herein is well calculated to fulfill the objects stated above, it will be appreciated that numerous modifications and embodiments can be devised by those skilled in the art. Therefore, it is intended that the appended claims cover all such modifications and embodiments as falling within the true spirit and scope of the present invention.

1. A process for the preparation of a protected compound, comprising: protecting the hydroxy group of 5,6-dihydro-4-(R)-hydroxy-6-(S)-methyl-4H-thieno-[2,3-b]thiopyran 7,7-dioxide, having the structural Formula II 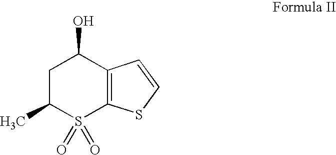
with a sulfonic acid derivative, in the presence of an organic base and a polar aprotic organic solvent, to obtain a protected derivative of the compound of Formula II.

2. The process of claim 1, wherein the sulfonic acid derivative is arylsulfonyl or alkylsulfonyl chloride.

3. The process of claim 2, wherein the arylsulfonyl chloride is benzylsulfonyl chloride, tosyl chloride or toluenesulfonyl chloride.

4. The process of claim 3, wherein the arylsulfonyl chloride is benzylsulfonyl chloride.

5. The process of claim 1, wherein the process is performed at a temperature of up to about 0° C.

6. The process of claim 5, wherein the process is performed at a temperature of from about −30° to about 0° C.

7. The process of claim 1, wherein the organic base is selected from the group consisting of pyridine, triethylamine, and N,N-diisipropylethylamine.

8. The process of claim 7, wherein the organic base is triethylamine.

9. The process of claim 1, wherein the polar aprotic organic solvent is selected from the group consisting of acetone, dioxane, acetonitrile, tetrahydrofuran, ethyl acetate, 2-methyltetrahydrofuran, and pyridine.

10. The process of claim 9, wherein the polar aprotic organic solvent is tetrahydrofuran or ethyl acetate.

11. A process for the preparation of a compound of Formula IV 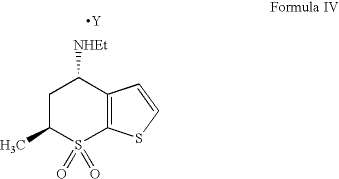
comprising: aminating a protected derivative of formula II, wherein Y is an acid moiety, with an alkyl amine and an acid in the presence of a solvent selected from the group consisting of a polar aprotic organic solvent, water and a mixture thereof, to give 5,6-dihydro-4-(S)-ethylamino-6-(S)-methyl-4H-thieno-[2,3-b]thiopyran 7,7-dioxide salt of Formula IV.

12. The process of claim 11, wherein the amination is carried out at a temperature of about 20° C. to about 30° C.

13. The process of claim 11, wherein the amination is carried out for about 16 hours to about 20 hours.

14. The process of claim 11, wherein the alkyl amine is ethyl amine.

15. The process of claim 11, wherein the acid is an organic acid or an inorganic acid.

16. The process of claim 15, wherein the organic acid is selected from the group consisting of acetic acid, fumaric acid, and tartaric acid.

17. The process of claim 15, wherein the inorganic acid is selected from the group consisting of sulfuric acid, hydrochloric acid, and hydrobromic acid.

18. The process of claim 17, wherein the inorganic acid is hydrochloric acid.

19. The process of claim 11, wherein the polar aprotic organic solvent is selected from the group consisting of acetone, dioxane, acetonitrile, tetrahydrofuran, ethyl acetate, 2-methyltetrahydrofuran, and pyridine.

20. The process of claim 19, wherein the polar aprotic organic solvent is tetrahydrofuran or ethyl acetate.

21. A process for preparing dorzolamide salt of formula I 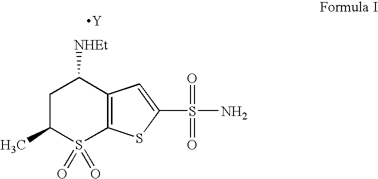
comprising: sulfonamidating of the compound of Formula IV, wherein Y is an acid moiety, by combining the compound of Formula IV with fuming sulfuric acid or chlorosulfonic acid, chlorinating the sulfonylated intermediate by the addition of inorganic chlorinated agent, evaporating the unreacted inorganic chlorinated agent from the reaction mixture, adding a polar aprotic organic solvent, adding a base and afterwards adding an acid corresponding to Y until dorzolamide salt compound of Formula I is obtained.

22. The process of claim 21, wherein the sulfonylation is at a temperature of about −10° C. to about 25° C.

23. The process of claim 21, wherein the sulfonylation is for about 2 to about 24 hours.

24. The process of claim 21, wherein the inorganic chlorinated agent is selected from the group consisting of thionyl chloride, SO₂Cl₂, PCl₃, and POC 3.

25. The process of claim 21, wherein the inorganic chlorinated agent is added at a temperature of from about −10° to about 25° C.

26. The process of claim 21, wherein after the addition of the inorganic chlorinated agent, the reaction mixture is heated to a temperature of about 60° C. to about 65° C.

27. The process of claim 21, wherein the polar aprotic organic solvent is selected from the group consisting of acetone, dioxane, acetonitrile, tetrahydrofuran, ethyl acetate, 2-methyltetrahydrofuran, and pyridine.

28. The process of claim 27, wherein the polar aprotic organic solvent is tetrahydrofuran or ethyl acetate.

29. The process of claim 21, wherein the base is an organic base or an inorganic base.

30. The process of claim 29, wherein organic base is ammonia.

31. The process of claim 29, wherein the inorganic base is selected from the group consisting of NaOH, KOH, K₂CO₃, and Na₂CO₃.

32. The process of claim 21, wherein the base is added at a temperature of about −15° C. to about 30° C.

33. The process of claim 21, wherein after the addition of the polar aprotic organic solvent, the reaction mixture is added to the base.

34. The process of claim 21, wherein the dorzolamide salt compound of Formula I is dorzolamide HCl, of Formula I,

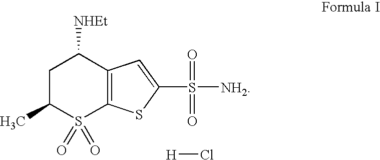

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. A process for the preparation of dorzolamide salt of structural Formula I, 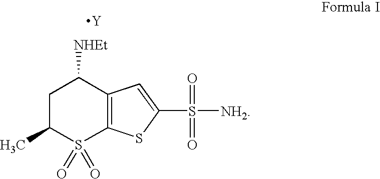
wherein Y is an acid moiety, comprising:
(a) protecting the hydroxy group of 5,6-dihydro-4-(R)-hydroxy-6-(S)-methyl-4H-thieno-[2,3-b]thiopyran 7,7-dioxide, having the structural Formula II 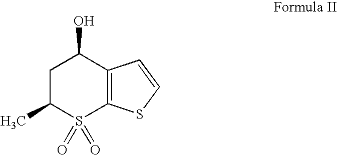
with a sulfonic acid derivative, in the presence of an organic base and a polar aprotic organic solvent, to obtain a protected derivative;
(b) aminating the protected derivative of formula II with an alkyl amine and an organic salt in the presence of a solvent selected from the group consisting of a polar aprotic organic solvent, water and a mixture thereof, to give 5,6-dihydro-4-(S)-ethylamino-6-(S)-methyl-4H-thieno-[2,3-b]thiopyran 7,7-dioxide salt of Formula IV; 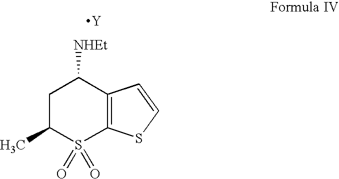
and
(c) sulfonamidating of the compound of Formula IV by combining the compound of Formula IV with fuming sulfuric acid or chlorosulfonic acid, chlorinating the sulfonylated intermediate by the addition of inorganic chlorinated agent, evaporating the inorganic chlorinated agent from the reaction mixture, adding a polar aprotic organic solvent, adding a base and afterwards adding an acid until dorzolamide salt compound of Formula I is obtained;
(d) purifying the dorzolamide salt compound of Formula I; and
(e) recovering the dorzolamide salt compound of Formula I.

54. The process of claim 53, wherein the sulfonic acid derivative in step (a) is arylsulfonyl or alkylsulfonyl chloride.

55. The process of claim 54, wherein the arylsulfonyl chloride is benzylsulfonyl chloride, tosyl chloride or toluenesulfonyl chloride.

56. The process of claim 55, wherein the arylsulfonyl chloride is benzylsulfonyl chloride.

57. The process of claim 53, wherein step (a) is performed at a temperature of up to about 0° C.

58. The process of claim 57, wherein step (a) is performed at a temperature of from about −30° to about 0° C.

59. The process of claim 53, wherein the base in step (a) is selected from the group consisting of pyridine, triethylamine, and N,N-diisipropylethylamine.

60. The process of claim 53, wherein the base in step (a) is triethylamine.

61. The process of claim 53, wherein the polar aprotic organic solvent in step (a) is selected from the group consisting of acetone, dioxane, acetonitrile, tetrahydrofuran, ethyl acetate, 2-methyltetrahydrofuran, and pyridine.

62. The process of claim 61, wherein the polar aprotic organic solvent tetrahydrofuran or ethyl acetate.

63. The process of claim 53, wherein the amination in step (b) is carried out at a temperature of about 20° C. to about 30° C.

64. The process of claim 53, wherein the amination in step (b) is carried out for about 16 hours to about 20 hours.

65. The process of claim 53, wherein the alkyl amine in step (b) is ethyl amine.

66. The process of claim 53, wherein the acid in step (b) is an organic acid or an inorganic acid.

67. The process of claim 66, wherein the organic acid is selected from the group consisting of acetic acid, fumaric acid, and tartaric acid.

68. The process of claim 66, wherein the inorganic acid is selected from the group consisting of sulfuric acid, hydrochloric acid, and hydrobromic acid.

69. The process of claim 68, wherein the inorganic acid is HCl.

70. The process of claim 53, wherein the polar aprotic organic solvent in step b) is selected from the group consisting of acetone, dioxane, acetonitrile, tetrahydrofuran, ethyl acetate, 2-methyltetrahydrofuran, and pyridine.

71. The process of claim 70, wherein the polar aprotic organic solvent is tetrahydrofuran or ethyl acetate.

72. The process of claim 53, wherein the compound of Formula IV in step (c) undergoes sulfonylation at a temperature of about −10° C. to about 25° C.

73. The process of claim 53, wherein the sulfonylation in step (c) is for about 2 to about 24 hours.

74. The process of claim 53, wherein the chlorination in step (c) is at a temperature of from about −10° to about 25° C.

75. The process of claim 53, wherein the inorganic chlorinated agent in step (c) is selected from the group consisting of thionyl chloride, SO₂Cl₂, PCl₃, and POCl₃.

76. The process of claim 53, wherein after the addition of the inorganic chlorinated agent in step c), the reaction mixture is heated to a temperature of about 60° C. to about 65° C.

77. The process of claim 53, wherein the polar aprotic organic solvent in step (c) is selected from the group consisting of acetone, dioxane, acetonitrile, tetrahydrofuran, ethyl acetate, 2-methyltetrahydrofuran, and pyridine.

78. The process of claim 77, wherein the polar aprotic organic solvent is tetrahydrofuran or ethyl acetate.

79. The process of claim 53, wherein the base in step (c) is an organic base or an inorganic base.

80. The process of claim 79, wherein the organic base is ammonia.

81. The process of claim 79, wherein the inorganic base is selected from the group consisting of NaOH, KOH, K₂CO₃, and Na₂CO₃.

82. The process of claim 51, wherein the base in step (c) is added at a temperature of about −15° C. to about 30° C.

83. The process of claim 53, wherein after the addition of the polar aprotic organic solvent, the reaction mixture is added to the base.

84. The process of claim 53, wherein the purification in step (d) of the dorzolamide salt comprises: dissolving the dorzolamide salt in water, adding a base until a basic slurry is obtained, extracting the basic slurry with aprotic polar organic solvent, which is immiscible in water, until two phases are obtained, separating the organic phase, concentrating the organic phase to obtain a residue of dorzolamide base, cooling the residue, combining the residue with an acid and with a polar aprotic organic solvent to obtain an acidic slurry, cooling the slurry to obtain a precipitate of dorzolamide salt, and recovering the dorzolamide salt.

85. (canceled)

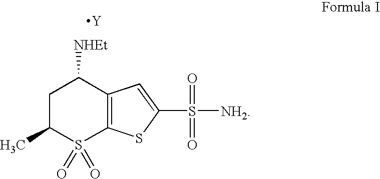

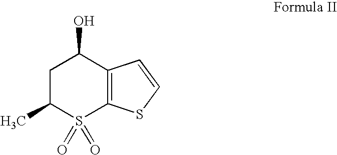

86. The process of claim 1, further comprising converting the protected compound of formula II to a compound of formula I.

87. The process of claim 11, further comprising converting the compound of formula IV to a compound of formula I.
